(19) United States
(12) Patent Application Publication
(10) Pub. No.: US 2006/0281822 A1
(43) Pub. Date: Dec. 14, 2006

(54) Treatment and prevention of elevated homocysteine

(75) Inventor: Jeremy E. Appleton, Portland, OR (US)

Correspondence Address: MCANDREWS HELD & MALLOY, LTD, 500 WEST MADISON STREET, SUITE 3400, CHICAGO, IL, 60661, US

(73) Assignee: Cardinal Associates, Inc.

(21) Appl. No.: 11/407,715

(22) Filed: Apr. 20, 2006

Related U.S. Application Data
(60) Provisional application No. 60/673,051, filed on Apr. 20, 2005.

(51) Int. Cl. A61K 31/10

(52) U.S. Cl. 514/709

(57) ABSTRACT

A method for reducing elevated levels of homocysteine in a person comprising the steps of measuring the level of homocysteine in a person; detecting an elevated level of homocysteine in the person; and responsive to said step of detecting, administering to said person a nutritional supplement comprising dimethyl sulfone.

### RELATED APPLICATIONS

Not applicable.

### Field of the Invention

This invention relates to the use of dimethyl sulfone preparations and methods for using such preparations for supporting normal homocysteine levels and for preventing and treating elevated homocysteine levels in the blood. More particularly, it relates to the administration of dimethyl sulfone by multiple routes, including oral, intravenous, topical, and other routes to reduce homocysteine and to maintain normal homocysteine levels.

The invention also relates to methods for using dimethyl sulfone in combination with other nutritional ingredients, dietary supplements, excipients, foods, beverages, food additives, and drugs for maintenance of normal homocysteine levels.

### BACKGROUND

Homocysteine is an amino acid formed in the body. It is created by the breakdown of another amino acid, methionine. High dietary consumption of methionine, which can be found in meats and dairy products, can result in the overproduction of homocysteine.

Homocysteine appears to be a nerve and vessel toxin, promoting mortality and disease. If the right cofactors are present, homocysteine will eventually convert to cysteine and other beneficial compounds. If the cofactors are lacking, homocysteine will build up to toxic levels.

FIG. 1 shows that homocysteine is metabolized in the body through one of two possible pathways—remethylation or transsulfuration. Remethylation is a process that utilizes folate, vitamin B-12 or betaine (trimethylglycine) to convert homocysteine back to methionine. Alternately, transsulfuration utilizes vitamin B6, pyridoxal-5-phosphate, to catabolize excess homocysteine into a number of metabolites for eventual excretion from the body.

Normal homocysteine levels are from 2.2 to 13.2 μmol/l. Loehrer, et al, Influence of oral S-adenosylmethionine on plasma 5-methyltetrahydrofolate, S-adenosylhomocysteine, homocysteine, and methionine in healthy humans. J Pharmacol Exp Ther., 1997 August; 282(2):845-50. Levels of homocysteine in typical Western populations are about 12 μmol/l. Lowering blood homocysteine with folic acid based supplements: meta-analysis of randomized trials. Homocysteine Lowering Trialists' Collaboration, BMJ. 1998 March 21;316(7135):894-8. Although this is “normal,” it is not necessarily healthy.

Research shows that elevated serum levels of homocysteine are a major cause of cardiovascular disease and cerebrovascular disease. Cardiovascular disease includes ischemic heart disease (heart attack), coronary artery disease (plaque obstruction of the coronary arteries to the heart), and stroke. Elevated homocysteine levels are believed to damage coronary arteries or make it easier for platelets to clump together and form a clot. Studies have shown that high serum homocysteine-related blood vessel damage may account for up to 20% of heart attacks, 40% of strokes, and 60% of peripheral venous occlusions in the United States. Further, a meta-analysis conducted in 2002 concluded that a 5 μmol/l increase in homocysteine increased the risk of cardiovascular disease by 23% and of stroke by 42%.

Increasing evidence suggests that homocysteine is also associated with cognitive impairment and Alzheimer's disease. Homocysteine is, in fact, toxic to the medulloblastoma cells of the brain. This cell type may be involved in the degenerative processes of Alzheimer's and Parkinson's. People with elevated levels of homocysteine have nearly double the risk of developing Alzheimer's disease, according to a report from researchers at Boston University. The findings, which come from the long-running Framingham Study, are the first to tie homocysteine levels measured several years before with later diagnosis of Alzheimer's and other dementias.

Elevated homocysteine levels have also been associated with cognitive impairment; depression; chronic fatigue syndrome; rheumatoid arthritis; adverse outcomes in pregnancy, including premature births, preeclampsia, stillbirths, and birth defects (e.g., spina bifida, other neural tube defects, congenital heart defects); spontaneous abortion (miscarriage); schizophrenia; multiple sclerosis; osteoporosis; gastritis; renal diseases including renal failure, renal transplant, and uremia; oxidative stress; inflammation; elevated inflammatory mediators; eye disorders including nonarteritic anterior ischemic optic neuropathy and retinal venous occlusive disease; and cancer.

Oral vitamin formulations combining vitamin B-12, folic acid, and vitamin B-6 have traditionally been used in the treatment of elevated serum levels of homocysteine. However, many people suffer from conditions caused or exacerbated by, or associated with, elevated homocysteine levels and do not receive treatment since the symptoms of these diseases are not easily recognized. Thus, by the time a person finally suffers recognizable symptoms of one of these diseases or are diagnosed, the severity of the disease may have become life-threatening.

Homocysteine levels are currently measured through the use of several different analytical methods: capillary gas chromography—mass spectrometry (GC-MS), liquid chromatography electrospray tandem mass spectrometry (LC-MS-MS), high-pressure liquid chromatography (HPLC) with photometric detection, HPLC with fluorometric detection, HPLC with electrochemical detection, and immunoassay.

The homocysteine concentration frequently correlates inversely with the folate concentration, since folate is a cofactor for the enzyme MTHFR that breaks down homocysteine. A detection of folate level in addition to the homocysteine level is therefore diagnostically appropriate. Since folate is mainly present and takes effect in the erythrocytes (approx. 98%), the detection of the erthyrocytic folate or total folate is more conclusive than the usual detection of the plasma folate or serum folate.

### SUMMARY OF THE METHOD

A primary object of the method is to provide a dietary supplement and a method of using same for controlling and maintaining normal serum levels of homocysteine.

A further primary object of the method is to provide a dietary supplement and a method of using same for treating or preventing diseases caused or exacerbated by elevated levels of homocysteine such as cardiovascular diseases, rheumatoid arthritis, Alzheimer's disease, adverse outcomes in pregnancy, renal diseases, eye disorders, and cancer.

One or more of the preceding objects, or one or more other objects which will become plain upon consideration of the present specification, are satisfied by the method described herein.

One aspect of the method, which satisfies one or more of the above objects, is accomplished by providing a therapeutically effective amount of dimethyl sulfone.

One aspect of the method, which satisfies one or more of the above objects, may be further accomplished by providing a formulation of dimethyl sulfone in combination with at least one other ingredient having homocysteine controlling properties.

The methods and compositions of the present method address the need in the art for an effective and convenient method for controlling and maintaining homocysteine levels. Thus, the present method provides a useful alternative to the current methods.

One aspect of the method provides a composition for treating or preventing vascular disease consisting of dimethyl sulfone in combination with nutrients or drugs intended to alter homocysteine.

According to a further aspect of the method provides a composition for treating or preventing vascular disease consisting of dimethyl sulfone in combination with one or more nutritional ingredients and drugs.

According to a further aspect of the method provides a composition for treating or preventing vascular disease consisting of dimethyl sulfone in combination with one or more excipients.

According to another aspect of the method provides a method of treating or preventing vascular disease includes periodically administering a therapeutically effective amount of dimethyl sulfone.

According to another aspect of the method, a method of treating or preventing vascular disease includes periodically administering a formulation including a therapeutically effective amount of dimethyl sulfone in combination with nutrients or drugs intended to alter homocysteine.

According to another aspect of the method, a method of treating or preventing vascular disease includes periodically administering a formulation including a therapeutically effective amount of dimethyl sulfone in combination with one or more nutritional ingredients and drugs.

According to another aspect of the method, a method of treating or preventing vascular disease includes periodically administering a formulation including a therapeutically effective amount of dimethyl sulfone in combination with one or more excipients.

The beneficial effects of the present method include the promotion and maintenance of homocysteine levels in the body.

Another beneficial effect of the present method is its ability to promote and maintain health. More particularly, many people suffer from conditions caused or exacerbated by, or associated with, elevated homocysteine levels such as vascular disease. Often these people do not receive treatment since the symptoms of these diseases are not easily recognized. Thus, by the time a person finally suffers recognizable symptoms of one of these diseases or are diagnosed, the severity of the disease may have become life-threatening.

The present method addresses this dilemma by providing means of treating and preventing conditions caused or exacerbated by, or associated with, elevated homocysteine levels. By providing the dietary supplement of the present method, one which has little or no side effects and is capable of addressing the causes and symptoms of conditions caused or exacerbated by, or associated with, elevated homocysteine levels, people are able to receive a treatment or a preventative for a disease or diseases from which they unknowingly suffer or are at risk from suffering.

It will be apparent to those skilled in the art that only the preferred embodiments have been described by way of exemplification and that there are various modifications which fall within the scope of this method.

### BRIEF DESCRIPTION OF THE DRAWINGS

FIG. 1 is a representation of the pathways by which homocysteine is metabolized.

FIG. 2 illustrates the results of the evaluation of dimethyl sulfone's potential activities conducted during the 2005 study, Kim, et al, Efficacy of methylsulfonylmethane (MSM) in osteoarthritis pain of the knee: a pilot clinical trial. OsteoArthritis and Cartilage, 2005 October.

### DETAILED DESCRIPTION OF THE METHOD

The method of the present invention contemplates the use of dimethyl sulfone as a methyl donor in the remethylation of homocysteine to methionine. Dimethyl sulfone (DMSO2, methylsulfone, methylsulfonylmethane, MSM) is a nutritional supplement comprising sulfur, methyl groups, and oxygen. Dimethyl sulfone may be administered by multiple routes, including oral, intravenous, and topical.

In 2005, a study was conducted to determine the efficacy of dimethyl sulfone in the treatment of pain and physical function impairment due to osteoarthritis. Patients were given dosage of 6 g of dimethyl sulfone (Distilled MSM microprill (OptiMSM®, Cardinal Nutrition, Vancouver, Wash.)) per day in a stepwise approach. In week 1, dosages started with 2 g/day in two divided doses for 3 days, and then increased to 4 g/day for 4 days. Week 2, increased to 6 g/day. In the control group, a placebo was administered that consisted of inert ingredients and that was indistinguishable in color, size and taste from the dimethyl sulfone. The study concluded that dimethyl sulfate administered in 3 g dosages twice a day improved symptoms of pain and physical function. Kim, et al, Efficacy of methylsulfonylmethane (MSM) in osteoarthritis pain of the knee: a pilot clinical trial. OsteoArthritis and Cartilage, 2005 October

During the 2005 osteoarthritis study, described above, dimethyl sulfone's potential activities were also evaluated. Serum homocysteine, high sensitive C-reactive protein (CRP), erythrocyte sedimentation rate (ESR), and urine malondialdehyde (MDA) were measured at baseline and 12 weeks.

Homocysteine is formed in the body. As described in detail above, hyperhomocysteinemia is associated with cardiovascular disease and other conditions; and reducing homocysteine with micronutrients has been demonstrated to decrease vascular disease.

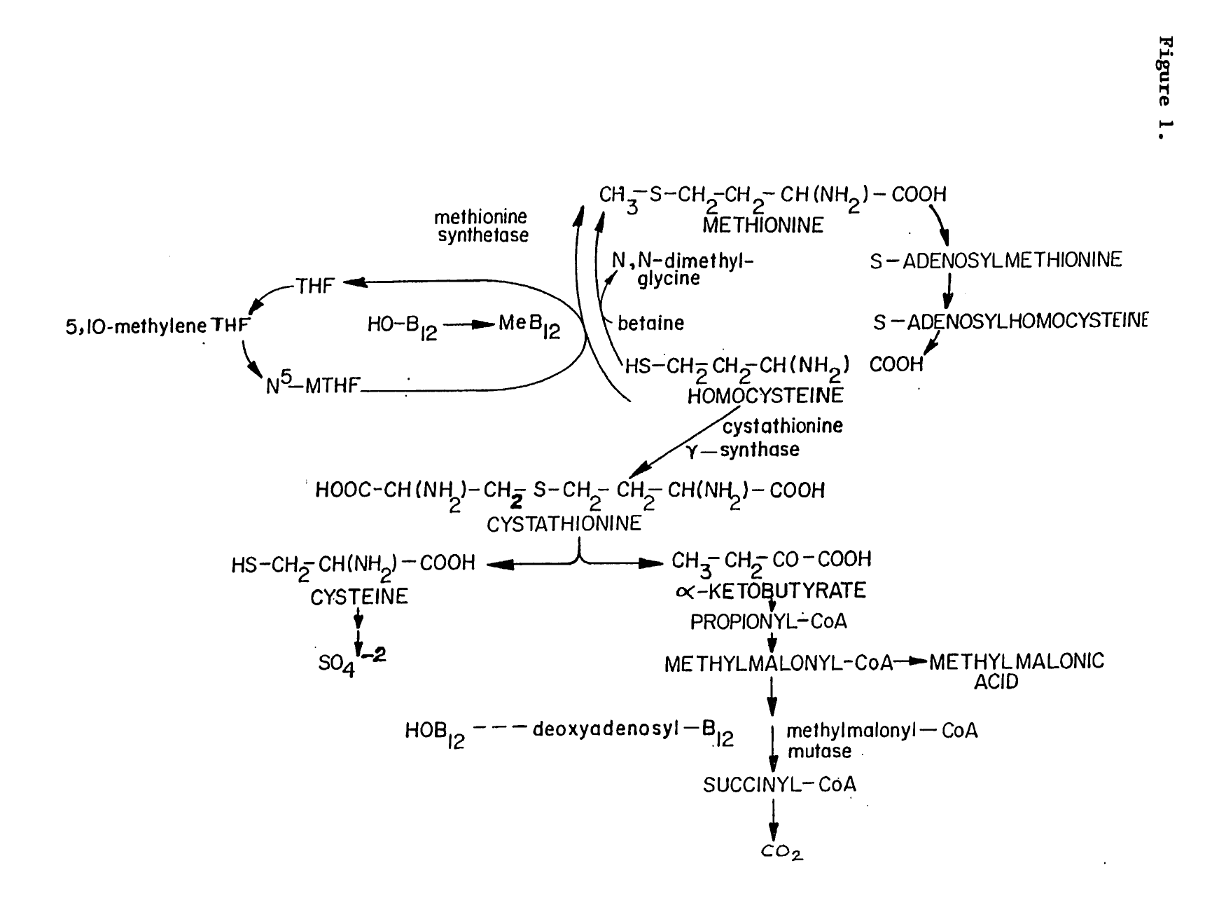

FIG. 1 shows that once homocysteine is produced it is metabolized in the body through one of two possible pathways—remethylation or transsulfuration. Remethylation is a process that utilizes folate, vitamin B-12 or betaine (trimethylglycine) to convert homocysteine back to methionine. During this process, folate acts as a methyl donor to facilitate the conversion of homocysteine to methionine.

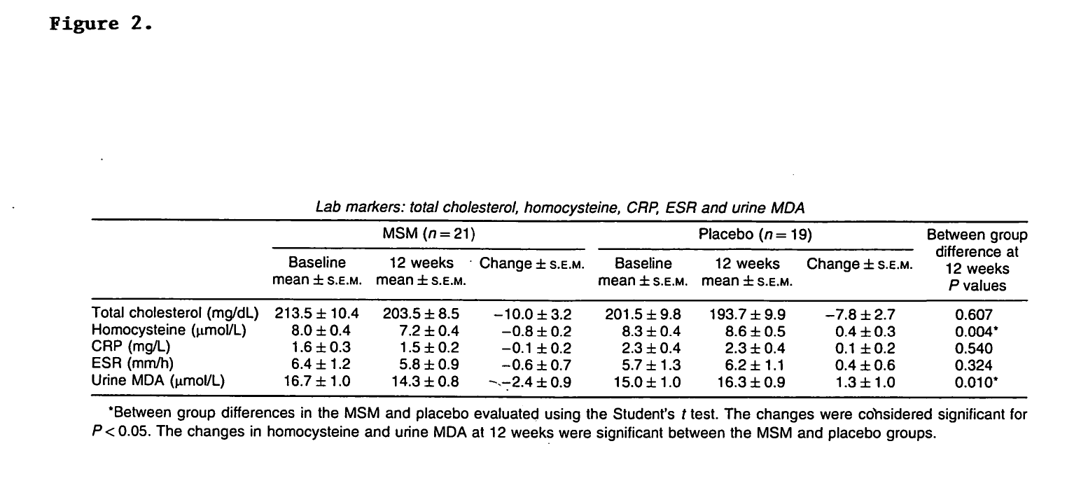

Since dimethyl sulfone is a putative methyl donor, it was believed that dimethyl sulfone may act as a co-factor in reducing homocysteine levels. FIG. 2 shows that during the 2005 osteoarthritis study homocysteine levels were significantly decreased in the group administered dimethyl sulfone. The decrease in homocysteine is believed to be due to the donation of dimethyl sulfone's two methyl groups. Folic acid and B vitamins are known to reduce hyperhomocysteinemia through similar mechanisms.

The combined decreases in homocysteine and urine MDA during the 2005 osteoarthritis study supports the role of dimethyl sulfone in metabolic processes requiring methylation, such as antioxidant capacities.

The method contemplates the use of dimethyl sulfone alone and in combination with other nutritional ingredients including supplements, excipients, foods, beverages, food additives, and drugs specifically chosen and combined according to their biological activities.

In one aspect of the method, a method of treating or preventing elevated homocysteine levels and, therefore, vascular disease, includes administering a formulation including a therapeutically effective amount of dimethyl sulfone in combination with nutrients or drugs intended to alter homocysteine selected from: the group consisting of the various forms of vitamin B-6 (e.g., pyridoxine), the various forms of vitamin B-12 (e.g., cyanocobalamin), the various forms of folic acid (e.g., pteroglutamic acid), betaine, andenosylmethionine, choline, and acetylcysteine.

According to another aspect of the method, a method of treating or preventing elevated homocysteine levels and, therefore, vascular disease, includes administering a formulation including a therapeutically effective amount of dimethyl sulfone in combination with one or more nutritional ingredients and drugs, each in a therapeutically effective amount, selected from: the various forms of vitamin B (e.g., B-1 (thiamine), B-2 (riboflavin), B-3 (niacin), B-5 (pantothenic acid)); Coenzyme Q10; the various forms of vitamin E (e.g., tocopherol); amino acids such as cysteine, arginine, camitine, 5-HTP, glutamic acid, glutamine, glycine, histidine, isoleucine, L-tyrosine, leucine, methionine, ornithine, phenylalanine, taurine, valine; anthocyanins, anthocynidins, anthocysanosides, and other antioxidant pigments; vitamin C (ascorbic acid) and its congeners; vitamin A and its congeners; carotenoids (e.g., beta-carotene, lutein, lycopene); xanthophylls; vitamin K and its congeners; vitamin D and its congeners; acetyl-L-camitine; alanine; agae (blue-green); aloe; androstenedine; bee pollen; bee propolis; beta-glucan; beta-sitosterol; betaine HCl; the various probiotics (e.g., acidophilus); the various bioflavonoids (e.g., quercetin and rutin); biotin; black currant seed oil; boric acid; boron; bovine cartilage; bovine colostrum; brewer's yeast; bromelain; calcium D-glucarate; carnosine; cartilage; the various cetylated fattyacids (e.g., cetyl meristoleate); chitosan; chlorella; chlorophyll; chondroitin sulfate; chondroitin/glucosamine combinations; chromium; coconut oil; cod liver oil; collagen; colloidal silver; conjugated linoleic acid; copper; coral calcium; creatine monohydrate; curcumin; D-mannose; daidzein; dehydroepiandrosterone (DHEA); docosahexaenoic acid (DHA); digestive enzymes; diindolylmethane (DIM); dimethyl sulfoxide (DMSO); dimethylaminoethanol; eicosapentaenoic acid; enzymes (e.g., lactase, protease, lipase, amylase); epigallocatechin gallate (EGCG); estrogens and phytoestrogens; evening primrose oil; the various forms of iron (e.g., ferrous sulfate); fiber; fish oil; fluoride; fructo-oligosaccharides (FOS); fumaric acid; gamma linolenic acid; gamma oryzanol; gamma-amino butyric acid; garcinia cambogia; garlic; genistein; ginko and its extracts; glandular extracts (e.g., adrenal, liver, spleen, thymus, thyroid, etc.); glucaric acid; glucomannan; glucosamine; glucosamine hydrochloride; glucosamine sulfate; GTF chromium; glutamic acid; glutamine; glutathione; glycine; grape seed extract; grapefruit seed extract; green tea; green-lipped mussel; huperzine A; hydrochloric acid; hydroxycitric acid; indole-3-carbinol; inosine; inositol hexaniacinate; inositol hexaphosphate; inulin oligosaccharides; iodine; ipriflavone; kelp; lecithin; lignan; linum usitatissimum; lipoic acid; lysine; magnesium; malic acid; manganese; mannose; medium chain triglycerides; melatonin; methoxyisoflavone; milk thistle and its extracts (e.g., silymarin); molybdenum; N-acetul-glucosamine; NADH; octacosanol; oligomeric proanthocyanidins; oligosaccharides; omega-3 fatty acids; ornithine alphaketoglutarate; palm kernel oil; palm oil; pancreatic enzymes; pancreatin; papain; para-aminobenzoic acid, phosphatidyl choline; phosphatidylserine; policosanol; pregnenolone; proanthocyanidins; progesterone; propionyl-L-camitine; protein (including soy and whey); psyllium; pyruvic acid; resveratrol; ribose; royal jelly; rutin; rye pollen; 7-KETO; saccharomyces boulardii; saccharomyces cerevisiae; selenium; shark cartilage; silica hydride; silicon; the various soy products (e.g., isoflavones); spirulina; starch blockers; strontium; sulforaphane; sulfur; thiotic acid; tyrosine; vanadium; vinacamine; vinpocetine; wheat grass; xylitol; and zinc.

According to another aspect of the method, a method of treating or preventing elevated homocysteine levels and, therefore, vascular disease, includes administering a formulation including a therapeutically effective amount of dimethyl sulfone in combination with one or more excipient selected from: silicon dioxide, stearic acid, cellulose, methylcellulose, ethylcellulose, microcrystalline cellulose, ascorbyl palmitate, crosscarmellose sodium, beeswax, benzyl alcohol, dicalcium phosphate, calcium phosphate, calcium sulfate, polyethylene glycol, locus bean products, maltodextrin, hydrogenated vegetable oil, colorants and dyes (both natural and artificial), natural and artificial flavors, sugars (e.g., glucose, fructose, sucrose, sorbitol, aspartame, high fructose corn syrup, etc.), gelatin, glycerin, fatty acid derivatives (e.g., glyceryl monostearate, etc.), hydroxypropyl methylcellulose phthalate, maltol, polyvinyl-pyrrolidone, potassium sorbate, phthalates, rice flour, rice powder, shellac, silica, talc, sodium benzoate, sodium carboxymethylcellulose, sodium lauryl sulfate, sorbitan mono-oleate, sorbitan tri-oleate, sunflower oil, titanium dioxide, and xanthan gum.

Each nutritional supplement, excipient, food, beverage, food additive, or drug component is selected as an ingredient in the administered composition for its ability to treat elevated homocysteine levels or to prevent elevated homocysteine levels.

The methods for combining dimethyl sulfone with the nutritional supplements, excipients, foods, beverages, food additives, and/or drugs of the present method are well known to those of ordinary skill in the art and may be accomplished at a number of commercial production laboratories around the world.

In a preferred embodiment of the method, a measurement of the level of homocysteine in a person is made. This is done by one of several different analytical methods: capillary gas chromography—mass spectrometry (GC-MS), liquid chromatography electrospray tandem mass spectrometry (LC-MS-MS), high-pressure liquid chromatography (HPLC) with photometric detection, HPLC with fluorometric detection, HPLC with electrochemical detection, or immunoassay.

Next, the homocysteine level is analyzed relative to a standard, for example, 10% above a normal level for this person. If the normal level for this person is 12 μmol/l, then an elevated level will be defined as 10% above the normal level, i.e., 13.2 μmol/l.

If the measured homocysteine level exceeds 13.2 μmol/l then an elevated level of homocysteine in the person is said to be detected. This act of detecting will vary from person to person depending on the normal level of homocysteine for that person.

In response to detecting an elevated level of homocysteine in the person, dimethyl sulfone is administered orally in the range of 1 mg up to 50,000 mg. It is expected that dosages in this range will be effective in the present invention. The dimethyl sulfone may be combined with other ingredients, as discussed above, including, for example, vitamin B-6, vitamin B-12, folic acid, and/or betaine.

In another preferred embodiment of the method, dimethyl sulfone is administered intravenously in the range of 1,000 mg up to 150,000 mg per day. It is expected that dosages in this range will be effective in the present invention.

Preferably, the compositions of the present method are prepared in a caplet dosage form, however it will be understood by those skilled in the art that other dosage forms may also be suitably prepared by known methods, for example, capsules, tablets, powders, pastes, liquids and similar dosage forms. Solid dosage forms for oral administration include caplets, capsules, tablets, pills, powders, and granules.

Liquid dosage forms for oral administration include pharmaceutically acceptable emulsions, solutions, suspensions, syrups, and elixirs.

Oral dosages may also be incorporated and administered in food and beverage products.

The compositions are preferably administered in spaced dosages throughout the day, for example, administered every twelve hours, so as to maintain the level of active ingredients in the system of the host.

What is claimed is:

1. A method for reducing elevated levels of homocysteine in a person comprising the steps of
measuring the level of homocysteine in a person;
detecting an elevated level of homocysteine in the person; and
responsive to said step of detecting, administering to said person an oral nutritional supplement comprising dimethyl sulfone wherein said supplement is administered in a daily dosage of dimethyl sulfone of about 1 to 50,000 mg.

2. The method of claim 1 and further including selecting said supplement to additionally contain nutritional ingredients other than dimethyl sulfone and said ingredients having the ability to control or maintain homocysteine levels.

3. The method according to claim 2 wherein said selecting includes selecting said supplement to contain one or more nutrients and drugs intended to alter homocysteine.

4. The method according to claim 2 wherein said selecting includes selecting said supplement to contain one or more nutritional ingredients and drugs.

5. The method according to claim 2 wherein said selecting includes selecting said supplement to contain one or more excipients.

6. The method of claim 1 wherein said step of administering is performed orally as a tablet, capsule, liquid, or powder for once or several times a day.

7. The method of claim 1 wherein said nutritional supplement is incorporated in and administered through a food or beverage product.

8. A method for reducing elevated levels of homocysteine in a person comprising the steps of
measuring the level of homocysteine in a person;
detecting an elevated level of homocysteine in the person; and
responsive to said step of detecting, administering to said person an intravenous nutritional supplement comprising dimethyl sulfone wherein said supplement is administered in a daily dosage of dimethyl sulfone of about 1,000 to 150,000 mg.

9. The method of claim 8 and further including selecting said supplement to additionally contain nutritional ingredients other than dimethyl sulfone and said ingredients having the ability to control or maintain homocysteine levels.

10. The method according to claim 9 wherein said selecting includes selecting said supplement to contain one or more nutrients and drugs intended to alter homocysteine.

11. The method according to claim 9 wherein said selecting includes selecting said supplement to contain one or more nutritional ingredients and drugs.

12. The method according to claim 9 wherein said selecting includes selecting said supplement to contain one or more excipients.

13. A method for aiding in preventing, delaying the onset of and/or slowing the progression of vascular disease in a person comprising the steps of
measuring the level of homocysteine in a person;
detecting an elevated level of homocysteine in the person; and
responsive to said step of detecting, administering to said person a nutritional supplement comprising dimethyl sulfone wherein said supplement is administered in a daily dosage of dimethyl sulfone of about 1 to 150,000 mg.

14. The method of claim 13 and further including selecting said supplement to additionally contain nutritional ingredients other than dimethyl sulfone and said ingredients having the ability to control or maintain homocysteine levels.

15. The method according to claim 14 wherein said selecting includes selecting said supplement to contain one or more nutrients and drugs intended to alter homocysteine.

16. The method according to claim 14 wherein said selecting includes selecting said supplement to contain one or more nutritional ingredients and drugs.

17. The method according to claim 14 wherein said selecting includes selecting said supplement to contain one or more excipients.

18. The method of claim 13 wherein said step of administering is performed orally as a tablet, capsule, liquid, or powder for once or several times a day.

19. The method of claim 13 wherein said step of administering is performed intravenously.

20. The method of claim 13 wherein said nutritional supplement is incorporated in and administered through a food or beverage product.

21. A method for aiding in preventing, delaying the onset of and/or slowing the progression of conditions caused or exacerbated by, or associated with, elevated homocysteine levels in a person comprising the steps of
measuring the level of homocysteine in a person;
detecting an elevated level of homocysteine in the person; and
responsive to said step of detecting, administering to said person a nutritional supplement comprising dimethyl sulfone wherein said supplement is administered in a daily dosage of dimethyl sulfone of about 1 to 150,000 mg.

22. The method of claim 21 and further including selecting said supplement to additionally contain nutritional ingredients other than dimethyl sulfone and said ingredients having the ability to control or maintain homocysteine levels.

23. The method according to claim 22 wherein said selecting includes selecting said supplement to contain one or more nutrients and drugs intended to alter homocysteine.

24. The method according to claim 22 wherein said selecting includes selecting said supplement to contain one or more nutritional ingredients and drugs.

25. The method according to claim 22 wherein said selecting includes selecting said supplement to contain one or more excipients.

26. The method of claim 21 wherein said step of administering is performed orally as a tablet, capsule, liquid, or powder for once or several times a day.

27. The method of claim 21 wherein said step of administering is performed intravenously.

28. The method of claim 21 wherein said nutritional supplement is incorporated in and administered through a food or beverage product.

29. The method according to claim 1 wherein said supplement is administered in a daily dosage of dimethyl sulfone of about 6,000 to 50,000 mg.

30. The method according to claim 1 wherein said supplement is administered in a daily dosage of dimethyl sulfone of about 6,000 mg.

31. The method according to claim 8 wherein said supplement is administered in a daily dosage of dimethyl sulfone of about 6,000 to 150,000 mg.

32. The method according to claim 8 wherein said supplement is administered in a daily dosage of dimethyl sulfone of about 6,000 mg.

33. The method according to claim 13 wherein said supplement is administered in a daily dosage of dimethyl sulfone of about 6,000 to 150,000 mg.

34. The method according to claim 13 wherein said supplement is administered in a daily dosage of dimethyl sulfone of about 6,000 mg.

35. The method according to claim 21 wherein said supplement is administered in a daily dosage of dimethyl sulfone of about 6,000 to 150,000 mg.

36. The method according to claim 21 wherein said supplement is administered in a daily dosage of dimethyl sulfone of about 6,000 mg.
